(19) United States
(12) Patent Application Publication
(10) Pub. No.: US 2016/0022818 A1
(43) Pub. Date: Jan. 28, 2016

(54) PHARMACEUTICAL FORMULATION COMPRISING LIPASE INHIBITOR HAVING INCREASED DISSOLUTION RATE AND REDUCED SIDE EFFECTS, AND METHOD FOR PREPARING SAME

(71) Applicant: UNIVERSITY-INDUSTRY FOUNDATION, YONSEI UNIVERSITY, Seoul (KR)

(72) Inventors: Sung-Joo HWANG, Seoul (KR); Kwang-Ho CHA, Gyeonggi-do (KR); Bokyung SUN, Incheon (KR); Han KANG, Incheon (KR); In-ho SONG, Gyeonggi-do (KR)

(21) Appl. No.: 14/776,357

(22) PCT Filed: Mar. 13, 2014
(86) PCT No.: PCT/KR2014/002115

§ 371 Date: Sep. 14, 2015

(30) Foreign Application Priority Data
Mar. 14, 2013 (KR) 10-2013-0027189

(51) Int. Cl. A61K 47/02; A61K 31/365

(52) U.S. Cl. CPC A61K 47/02; A61K 31/365

(57) ABSTRACT

The present invention provides a pharmaceutical formulation for increasing the dissolution rate of a lipase inhibitor and reducing side effects of the lipase inhibitor, including oily anal leakage, and a method for preparing the same, the pharmaceutical formulation comprising: a lipase inhibitor, and a porous adsorbent on which a thin film of the lipase inhibitor is formed.

### Technical Field

The present invention relates to a pharmaceutical formulation having an increased dissolution rate of a lipase inhibitor and reduced side effects and a method for preparing the same.

### BACKGROUND ART

Lipases are water-soluble enzymes which are secreted in gastric juice, pancreatic juice and intestinal juice, present in various types of tissues such as lungs, kidneys, adrenals, adipose tissues and placentas, and hydrolyze ester linkages in insoluble lipids. For example, human pancreatic lipases break down triglycerides, which are contained within dietary fats, into monoglycerides and fatty acids in the human digestive system, thereby helping the absorption of fats into bodies.

Meanwhile, lipase inhibitors refer to the compounds which can inhibit the actions of the lipases of the stomach, the pancreas, etc., and when such actions of the lipases are inhibited, the undecomposed triglycerides are not absorbed in the intestines but excreted in feces, resulting in the effects of inhibited absorption of fat and weight loss.

Such lipase inhibitors include lipstatin, orlistat, panclicins, hesperidin, ebelactones, esterastin, valilactone, etc.

Among them, orlistat is a derivative of lipstatin which is extracted from a bacterium Streptomyces toxytricini, and is a powerful lipase inhibitor. Orlistat has been found to inhibit lipase activities by binding to the activation sites of lipases inside a body, and the fact that orlistat is useful in inhibiting or preventing obesity and hyperlipidemia is disclosed in U.S. Pat. No. 4,598,089.

However, orlistat exhibits low solubility and dissolution rate in a biotic environment, thus having only a certain percentage (%) of drug molecules dissolve from the crystal; therefore, there is a problem in that it should be administered at a higher dose to achieve a sufficient level of biological activity. Thereupon, much research has been conducted to enhance the bio-availability by improving the solubility of orlistat, and Korean Unexamined Patent Application Publication No. 2009-0112508 discloses a formulation which contains orlistat having a particle size of 1 to 400 μm through grinding. However, even with the above literature, the dissolution rate at 10 minutes is higher by only about 1.4 times compared to the pre-existing formulation, and therefore, the solubility problem of orlistat, which is a poorly water-soluble compound, still remains.

In addition to the problem of poor water-solubility, when orlistat is administered, there is a problem of leakage of fat or oil through an anus due to the inhibited action of a lipase, or of the side effects such as oily spotting, oily/fatty stools and fecal urgency. Such side effects have become the factors that makes one strongly avoid the prescription of orlistat, and thus, much research has been conducted to develop a formulation having those side effects improved. However, until now, nothing has been known about a pharmaceutical formulation which shows both an improved dissolution rate of a lipase inhibitor such as orlistat and reduced side effects such as oily leakage.

### DISCLOSURE

### Technical Problem

Therefore, the inventors of the present invention would like to provide a pharmaceutical formulation which can simultaneously resolve the problems related to the solubility of such lipase inhibitors (which are poorly water-soluble drugs) and to the leakage of fat or oil through the anus; and a method of preparing the same.

### Technical Solution

In order to achieve the above-described objectives, the inventors of the present invention have conducted research on a new pharmaceutical formulation which can improve the pre-existing problems, namely, low solubility of lipase inhibitors and the side effects such as oily leakage. As a result, it was found that, through the formation of a thin film of a lipase inhibitor on a porous adsorbent, a dissolution rate of the lipase inhibitor can increase, and at the same time, the side effects, such as oily leakage, can be improved because the oil is re-adsorbed to the porous adsorbent which is then empty after the lipase inhibitor is dissolved, and so the present invention was completed.

Therefore, the present invention provides a method of preparing a pharmaceutical formulation for preventing and treating obesity, which aims at enhancing a dissolution rate and reducing the side effects, such as oily leakage, and includes the formation of a thin film of a lipase inhibitor on a porous adsorbent. The lipase inhibitor of the present invention is not limited to a certain type. For example, the lipase inhibitor may be selected from the group consisting of lipstatin, panclicins, hesperidin, ebelactones, esterastin, valilactone, orlistat, cetilistat, and the derivatives and pharmaceutically acceptable salts thereof. Preferably, the lipase inhibitor may be orlistat or cetilistat.

A thin film of a lipase inhibitor can be formed on a porous adsorbent through melting or dissolution of the lipase inhibitor and addition of the porous adsorbent. In the present invention, the order of melting or dissolving the lipase inhibitor and adding the porous adsorbent is not restricted. For example, one may form a thin film of a lipase inhibitor on a porous adsorbent through the addition of the porous adsorbent after melting or dissolving the lipase inhibitor, or may form a thin film of the lipase inhibitor on the porous adsorbent through mixing of the lipase inhibitor with the porous adsorbent followed by melting or dissolution.

In the present invention, the melting or dissolution of the lipase inhibitor is not limited to a certain method as long as it can liquefy the lipase inhibitor. In an embodiment of the present invention, one may melt a lipase inhibitor by heating or pressurizing it in the presence of a supercritical fluid. When heated or pressurized in the presence of a supercritical fluid, a melting point of the lipase inhibitor decreases, and thus, the lipase inhibitor melts. In the present invention, the supercritical fluid may be, although not limited to, supercritical carbon dioxide. For example, one may melt the lipase inhibitor by heating it to 40 to 50° C. and pressurizing it at 90 to 110 bars.

In an embodiment of the present invention, a lipase inhibitor can melt by being heated to a temperature greater than the melting point. For example, one may melt orlistat by mixing together orlistat and a porous adsorbent and heating them to 90° C. or more.

In an embodiment of the present invention, a lipase inhibitor can be dissolved through a solvent evaporation method using a volatile organic solvent. The volatile organic solvent may be, although not limited to, for example, methanol, ethanol, acetone, acetonitrile, dichloromethanol, propanol or mixtures thereof. Also the method may further comprise a process of removing the volatile organic solvent after dissolving a lipase inhibitor in a volatile organic solvent and adding a porous adsorbent to form a thin film of the lipase inhibitor on the porous adsorbent.

The method of preparing a pharmaceutical formulation according to the present invention does not use a non-volatile solvent, a solubilizer and a surfactant in melting or dissolving a lipase inhibitor. Non-volatile solvents, solubilizers and surfactants are substances commonly used to improve the solubility of a lipase inhibitor which is a poorly water-soluble drug. However, according to the present invention, one can improve the dissolution rate by resolving the solubility problem of a lipase inhibitor, even without using such substances. According to the present invention, one may form a porous thin film consisting only of a lipase inhibitor on a porous adsorbent either by melting the lipase inhibitor or by dissolving it in a volatile organic solvent and then removing the volatile organic solvent. When the lipase inhibitor is orlistat, the formation of a porous thin film can be confirmed through an observation under a scanning electron microscope, as illustrated in FIG. 1. As the result of observing the morphology of raw materials of pre-existing orlistat and that of the orlistat formulation of the present invention, it can be found that the formulation of the present invention forms a thin film which has a morphology which is new and different from that of the pre-existing orlistat formulation. Through the formation of such a thin film, a high dissolution rate can be secured as will be described hereinafter. Also, without solvents, solubilizers or surfactants being used, the occurrence of side effects which may result due to these additives can be prevented.

In an embodiment of the present invention, a porous adsorbent may be contained at 1 to 50 parts by weight with respect to 1 part by weight of a lipase inhibitor. For example, the porous adsorbent may be contained at 1 to 30 parts by weight, 1.5 to 15 parts by weight, 2 to 13 parts by weight, 3 to 10 parts by weight or 3 to 8 parts by weight with respect to 1 part by weight of the lipase inhibitor. Also, the porous adsorbent may be used without restriction as long as it is pharmaceutically acceptable. In an embodiment of the present invention, the porous adsorbent may be magnesium aluminometasilicate, a zeolite, MCM-41, SBA-15, light anhydrous silicic acid, magnesium aluminosilicate, Carbopol, a cellulose powder, crospovidone, sodium starch glycolate, croscarmellose sodium, carboxymethyl cellulose or a mixture thereof.

The present invention also provides a pharmaceutical formulation for preventing and treating obesity, which aims at enhancing dissolution rate and reducing the side effects such as oily leakage, contains a lipase inhibitor and a porous adsorbent, and has a thin film of the lipase inhibitor formed on the porous adsorbent.

The lipase inhibitor of the present invention is not limited to a certain type. For example, the lipase inhibitor may be selected from the group consisting of lipstatin, panclicins, hesperidin, ebelactones, esterastin, valilactone, orlistat, cetilistat, and the derivatives and pharmaceutically acceptable salts thereof. Preferably, the lipase inhibitor may be orlistat or cetilistat.

In an embodiment of the present invention, the pharmaceutical formulation having a lipase inhibitor as an active ingredient may contain the lipase inhibitor at 30 to 180 mg.

In an embodiment of the present invention, a porous adsorbent may be contained at 1 to 50 parts by weight with respect to 1 part by weight of a lipase inhibitor. The porous adsorbent is as described above.

The pharmaceutical formulation of the present invention which has a lipase inhibitor as an active constituent may be a formulation for oral administration. For example, it may be in capsules, tablets, coated tablets, granules or powders. As for a formulation for oral administration, the acceptable pharmaceutical carrier may include, although not limited to, a diluent, a preservative, a binder, a lubricant, a disintegrant, a swelling agent, a filler, a stabilizer, and combinations thereof. The carrier may also include all constituents of a coating composition which may include a plasticizer, a pigment, a colorant, a stabilizer and a superplasticizer. The examples of acceptable coating materials include cellulose polymers such as cellulose acetate phthalate, hydroxypropyl cellulose, hydroxypropyl methylcellulose, hydroxypropyl methylcellulose phthalate and hydroxypropyl methylcellulose acetate succinate; polyvinyl acetate phthalate, acrylic acid polymers and copolymers, methacrylic resins, zein, shellac and polysaccharides. In addition, the above coating materials may contain conventional carriers such as plasticizers, pigments, colorants, superplasticizers, stabilizers, foaming agents and surfactants. The optional additives which are pharmaceutically acceptable include diluents, binders, lubricants, disintegrants, pigments, stabilizers and surfactants.

Diluents are generally required to increase the volume of solid dosage formulations to the size suitable for the compression of tablets or the formation of beads and granules. Suitable diluents include, but are not limited to, dicalcium phosphate dihydrate, calcium sulfate, lactose, sucrose, mannitol, sorbitol, celluloses, microcrystalline celluloses, kaolin, sodium chloride, dried starches, hydrolysed starches, pregelatinized starches, silicon dioxide, titanium oxide, magnesium aluminum silicate and powdered sugars. Binders are used to give adhesive properties to solid dosage formations, in order to ensure that the tablets, beads or granules remain intact even after they are formed into dosage formulations. Suitable binders include starches, pregelatinized starches, gelatin, sugars (which include sucrose, glucose, dextrose, lactose and sorbitol), polyethylene glycol, waxes, acacia, tragacanth, natural and synthetic gums such as sodium alginate, celluloses including hydroxypropyl methylcellulose, hydroxypropyl cellulose, ethylcellulose and veegum, and synthetic polymers such as copolymers of acrylic acid and methacrylic acid, methacrylic acid copolymers, methyl methacrylate copolymers, aminoalkyl methacrylate copolymers, polyacrylic acid/polymethacrylic acid and polyvinylpyrrolidone.

Lubricants are used to facilitate the preparation of tablets. The examples of suitable lubricants include magnesium stearate, calcium stearate, stearic acid, glycerol behenate, polyethylene glycol, talc and mineral oils.

Disintegrants are used to facilitate the disintegration or breakup of dosage formations after administration, and they include starches, sodium starch glycolate, sodium carboxymethyl starches, sodium carboxymethyl cellulose, hydroxypropyl cellulose, pregelatinized starches, clays, celluloses, arginine, gums, or crosslinked polymers such as crosslinked PVP.

Stabilizers are used to inhibit or delay a decomposition reaction of a drug, for example, an oxidation reaction. The examples of suitable stabilizers include, but are not limited to: antioxidants, butylated hydroxytoluene (BHT), ascorbic acid, and salts and esters thereof; vitamin E, tocopherol, and salts thereof; sulfites such as sodium metabisulfite; cysteine and its derivatives; citric acid; propyl gallate and butylated hydroxyanisole (BHA).

As an example of a pharmaceutical formulation which contains a lipase inhibitor and is prepared through the formation of such a thin film having a new morphology, an orlistat formulation of the present invention, Xenical and a raw material of the pre-existing orlistat were subjected to a comparative dissolution test, and the results showed that the orlistat formulation of the present invention exhibited a significantly higher dissolution rate compared to Xenical (F. Hoffmann-La Roche, Ltd.) and the raw material of the pre-existing orlistat (FIG. 2). Also, the measured results of lipase activities showed that, because of its high rate of dissolution, the orlistat formulation of the present invention was effective in rapidly inhibiting lipase activity (FIG. 3). Based on these results, the formulation of Xenical currently available in the market and the formulation which contains the orlistat formulation of the present invention were orally administered to rats, and the triglyceride levels in blood were analyzed over time in order to assess the actual effectiveness in inhibiting fat breakdown in living bodies. The analyzed results showed that the group which was administered with the orlistat formulation of the present invention had low triglyceride levels in blood compared to the other group, which is an indication that the orlistat formulation of the present invention has excellent effectiveness in inhibiting fat breakdown (FIG. 4). Therefore, the pharmaceutical formulation of the present invention can use drugs in smaller amounts compared to what has been used in the pre-existing formulation, while still achieving equal or similar effectiveness.

The orlistat formulation of the present invention was tested by using rats as the model to confirm if the side effects such as oily leakage are reduced in vivo, which is the other purpose of the present invention. The raw materials of orlistat, the formulation of Xenical currently available in the market and the formulation of the present invention were orally administered, and the number of individuals experiencing oily leakage from their anuses was evaluated. The results showed that, as can be seen in Test Example 5, in the case of the orlistat formulation of the present invention, the number of individuals having oily leakage was much lower compared to the number of rat individuals showing oily leakage after administration of the formulation of Xenical currently available in the market. This indicates that the formulation of the present invention significantly reduces the side effects such as oily leakage. Likewise in the experimental results on humans as in Test Example 6, the oily leakage was found to be significantly reduced in the experimental group which was administered with the orlistat formulation of the present invention, as compared with the experimental group which was administered with Xenical.

### Advantageous Effects

The pharmaceutical formulation containing the lipase inhibitor of the present invention as an active constituent can resolve the solubility problem of the poorly water-soluble lipase inhibitor through the formation of a thin film of the lipase inhibitor on a porous adsorbent, and, at the same time, it can reduce the side effects such as oily or fatty anal leakage of the pre-existing formulation by having free oil reabsorbed into the porous adsorbent which is then empty after releasing the lipase inhibitor in the gastrointestinal tract.

### DESCRIPTION OF DRAWINGS

FIG. 1 is a scanning electron microscope image of a pharmaceutical formulation containing a lipase inhibitor which is prepared according to the present invention (a: raw material of orlistat in Comparative Example 1, b: raw material of Neusilin US2, c: Example 2-B).

FIG. 2 shows the results of a comparative dissolution test between the orlistat formulations from an embodiment of the present invention and Comparative Examples.

FIG. 3 shows the experimental results regarding lipase activity with the orlistat formulations from an embodiment of the present invention and with Comparative Examples.

FIG. 4 shows the results of change in triglyceride levels in the blood of a SD-rat model, to which the orlistat formulations from an embodiment of the present invention and Comparative Examples are administered.

### MODE FOR INVENTION

Hereinafter, the present invention will be described in detail through examples. The following examples are merely provided to illustrate the present invention, and the scope of the present invention is not limited to the following examples. The examples are provided to complete the disclosure of the present invention and to fully disclose the scope of the present invention to those of ordinary skill in the art, and the present invention is only defined by the range of the appended claims.

### COMPARATIVE EXAMPLE 1

A raw material of orlistat (Biocon Co., Ltd., India)

### COMPARATIVE EXAMPLE 2

The formulation of Xenical manufactured by F. Hoffmann-La Roche, Ltd., which is currently available in the market

### EXAMPLE 1

### Adsorption/Coating Processes using Supercritical Fluid

Orlistat and a porous adsorbent (Neusilin UFL2; Fuji chemical, Japan) were sufficiently mixed in accordance with each composition ratio of 1-A to 1-H and Comparative Example 3 as shown in Table 1 below, placed in a high pressure vessel and then sealed. Then the high pressure vessel was heated and, with a CO₂ pump, pressurized according to the temperature/pressure conditions of each composition. In this case, the melting point of the orlisat is lowered by supercritical CO₂ (Hanmi Gas, Co., Ltd.), and thereby all of orlistat melts as shown in Table 2. The melted orlistat is adsorbed/coated onto the porous adsorbent with the help of the supercritical fluid. Such a state was maintained for 90 minutes, and then the temperature and pressure were gradually reduced over 30 minutes, and then the adsorption/coating process was completed.

TABLE 1
Processing conditions and compositions of supercritical
adsorption/coating

| | Condition | | | | |
|---|---|---|---|---|---|
| | | | | Porous | |
| | | | | adsorbent: | Dissolution |
| | Temperature | Pressure | Orlistat | Neusilin | rate at 10 |
| Composition | (° C.) | (bar) | (%) | (%) | minutes % |
| 1-A | 35 | 80 | 54 | 46 | 58.7 |
| 1-B | 35 | 120 | 54 | 46 | 51.8 |
| 1-C | 45 | 80 | 54 | 46 | 60.5 |
| 1-D | 45 | 120 | 54 | 46 | 63.4 |
| 1-E | 55 | 80 | 54 | 46 | 56.7 |
| 1-F | 55 | 120 | 54 | 46 | 63.2 |
| 1-G | 45 | 100 | 20 | 80 | 84.5 |
| 1-H | 45 | 100 | 40 | 60 | 82.5 |
| Comparative | — | — | — | — | 7.0 |
| Example 1 | | | | | |
| Comparative | — | — | — | — | 7.9 |
| Example 2 | | | | | |
| Comparative | 45 | 100 | 60 | 40 | 46.4 |
| Example 3 | | | | | |

TABLE 2
Change in melting temperature of orlistat with pressure

| Pressure (bar) | Melting temperature of orlistat |
|---|---|
| 23 | 40 |
| 32 | 35 |
| 37 | 30 |
| 40 | 25 |
| 43 | 20 |
| 48 | 15 |

### EXAMPLE 2

### Supercritical Adsorption/Coating Processes using Various Porous Adsorbents

Orlistat was adsorbed onto a variety of adsorbents, namely, Neusilin® UFL2 (Fuji chemical, Japan), Neusilin US2 (Fuji chemical, Japan), MCM-41 (Simna Co., Ltd., United States), SBA-15, Aerosil (Tianjin Yinzhong Chemical Co. Ltd) and a zeolite (shijiazhuang Hejia Chemicals), by using a supercritical adsorption process. SBA-15 was synthesized and used according to the method described in Jana, S. K., 2004. Pore size control of mesoporous molecular sieves using different organic auxiliary chemicals. Microporous Mesoporous Mater. 68, 133-142. The process was carried out in conditions of 45° C. and 100 bars.

TABLE 3
Supercritical adsorption/coating processes using various porous adsorbents

| | Condition | | | | | |
|---|---|---|---|---|---|---|
| | Tem- | | | Porous | | Dissolution |
| | pera- | Pres- | Orli- | adsor- | Type of | rate at |
| | ture | sure | stat | bent | porous | 10 minutes |
| Composition | (° C.) | (bar) | (%) | (%) | adsorbent | % |
| 2-A | 45 | 100 | 40 | 60 | Neusilin ® | 82.5 |
| | | | | | UFL2 | |
| 2-B | 45 | 100 | 40 | 60 | Neusilin ® | 58.2 |
| | | | | | US2 | |
| 2-C | 45 | 100 | 40 | 60 | MCM-41 | 32.6 |
| 2-D | 45 | 100 | 40 | 60 | SBA-15 | 72.3 |
| 2-E | 45 | 100 | 40 | 60 | Aerosil | 65.7 |
| 2-F | 45 | 100 | 40 | 60 | Zeolite | 70.9 |
| Comparative | — | — | — | — | — | 7.0 |
| Example 1 | | | | | | |
| Comparative | — | — | — | — | — | 7.9 |
| Example 2 | | | | | | |

### EXAMPLE 3

### Adsorption/Coating Process using Melting Method and Solvent Evaporation Method

Orlistat:Neusilin in a ratio of 40:60 was adsorbed/coated on a porous adsorbent by using a melting method and a solvent evaporation method.

In the melting method, orlistat and Neusilin were mixed thoroughly, heated at 90° C. to melt the orlistat, and then adsorbed/coated onto the porous adsorbent (Example 3-A).

In the solvent evaporation method, orlistat was dissolved in ethanol (Samchun Pure Chemical Co., Ltd., Korea), a porous adsorbent was suspended in the orlistat solution, and then ethanol was evaporated to adsorb/coat the orlistat onto the porous adsorbent (Example 3-B).

### TEST EXAMPLE 1

Morphology Observation Test Through Scanning Electron Microscope

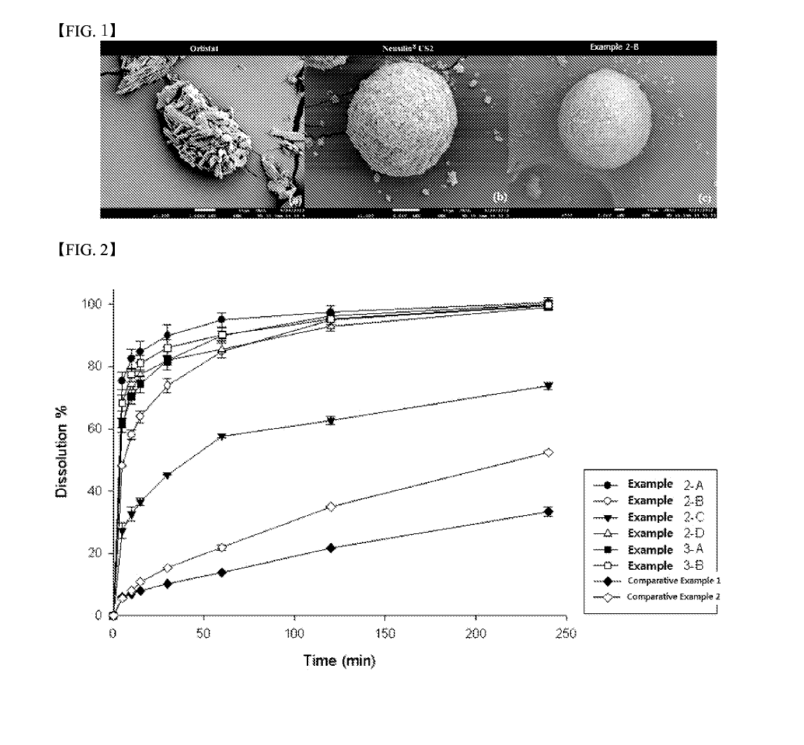

Morphologies of Examples and of Comparative Example 1 were observed through a scanning electron microscope (JSM-7000F, JEOL, Japan) (FIG. 1). The result was that, in contrast to the case of Comparative Example 1 in which needle-shaped crystals of several tens of micrometer (μm) appeared to be entangled like a skein of thread, the raw material of orlistat was not seen in the case of Example 2-B with the orlistat being adsorbed/coated onto the inner pore or surface of the porous adsorbent. This suggests that a thin film of the orlistat has formed on the porous adsorbent.

### TEST EXAMPLE 2

Comparative Dissolution Test

A comparative dissolution test was performed on the compositions prepared by Examples 1 to 3 and on the compositions prepared by Comparative Examples 1 and 2. The condition of dissolution was achieved with 900 ml of a 1% sodium lauryl sulfate solution (Duksan Pure Chemicals Co., Ltd., Korea) and a paddle method at 75 rpm, and the concentration of orlistat was quantified by HPLC-UV.

The result as plotted in FIG. 2 was that, when orlistat was adsorbed/coated onto a porous adsorbent, the dissolution rate was observed to be much higher as compared to Comparative Examples 1 and 2.

### TEST EXAMPLE 3

In-Vitro Lipase Inhibition Test

A lipase inhibition test was quantified by using p-nitrophenyl palmitate (p-NPP; Sigma Co., Ltd., United States) on the basis of the principle that p-NPP has its ester linkages cleaved by a lipase to be broken down into palmitic acid and p-nitrophenyl (ref. Dolenc et al., 2010. Nanosized particles of orlistat with enhanced invitro dissolution rate and lipase inhibition. Int. J. Pharm. 396, 149-155).

That is, 78 μl of an enzyme reaction solution, 20 μl of a lipase suspension (extracted from a pig, Sigma Co., Ltd., United States) and 2 μl of a comparative dissolution test solution were added to a 96-well to be incubated for 20 minutes, and then again, 100 μl of p-NPP test solution was added and the absorbance was measured at 405 nm to evaluate the lipase activity.

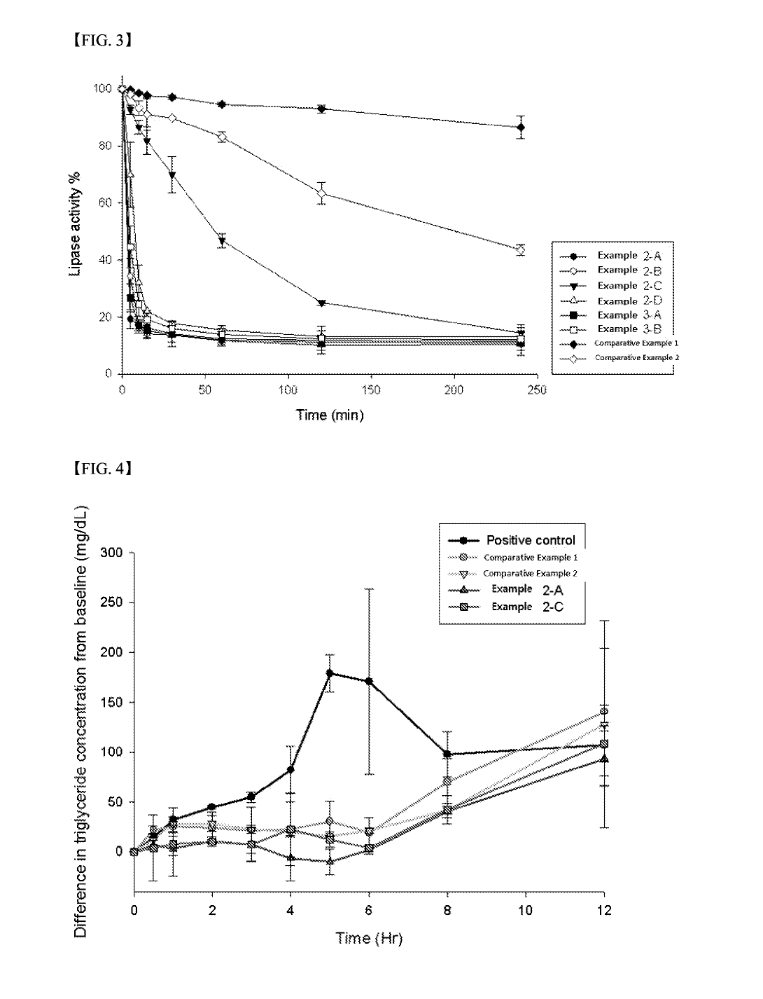

As the result of a lipase activity test, it was found as plotted in FIG. 3 that the lipase activity was rapidly inhibited, when orlistat was adsorbed/coated onto a porous adsorbent.

### TEST EXAMPLE 4

Measurement of Triglyceride Levels in Blood by using SD-Rat Model

Using 7-week old SD-rats (Samtaco Bio Korea Inc.), 1 ml of an olive oil (Samchun Pure Chemical Co., Ltd., Korea) was administered to each group and a drug suspended in a 0.25% HPMC (hydroxypropylmethyl cellulose) solution (Shin-Etsu chemical, Japan) was orally administered to the rats (corresponds to 2.5 mg of orlistat per kg of the body weight). The control group was administered with distilled water instead of the drug suspension. The blood of rats was collected for a fixed time interval, the triglyceride levels in the blood were measured by Cleantech TG-S of Asan Pharmaceutical, and, with the triglyceride level in the blood before testing used as a standard, changes in triglyceride levels in the blood after administration of an olive oil and drugs were plotted in FIG. 4 and Table 4. The result shows that, in contrast to the case of a positive control group without drug administration, in which olive oil was absorbed to cause the triglyceride level in the blood to rapidly increase, the drug administered groups (Comparative Example 1, 2 and Example 2-A) did not show an increase in the triglyceride level in blood. Moreover, Examples 2-A to 2-C showed much higher efficacy compared to Comparative Examples 1 and 2.

TABLE 4
Pharmacokinetic parameter

Pharmacokinetic parameter

| | Cₘₐₓ | AUC_{0->12 h} |
|---|---|---|
| Formulations | (mg/dl) | (mg hr/dl) |
| Positive control | 179 ± 18.9 | 1149.2 ± 219.4 |
| Comparative Example 1 | 140.7 ± 64.1 | 648.0 ± 203.2 |
| Comparative Example 2 | 128.0 ± 54.0 | 526.4 ± 138.9 |
| Example 2-A | 93.5 ± 27.5 | 345.9 ± 120.3 |
| Example 2-C | 108.7 ± 18.3 | 410.2 ± 145.2 |

### TEST EXAMPLE 5

Oily Leakage Test using SD-Rat Model

7-week old SD-rats were used, 0.5 ml of an olive oil was administered for each group, and a drug was suspended in a 0.25% HPMC solution to be orally administered to the rats (corresponds to 5 mg of orlistat per kg of the body weight). 6 hours later, the evaluation was assessed through the number of rats having oily leakage from their anuses.

TABLE 5
Oily leakage test using SD-Rat model

| | | Population having oily |
|---|---|---|
| | Population (rats) | leakage (rats) |
| Positive control | 26 | 0 |
| Comparative Example 1 | 26 | 17 |
| Comparative Example 2 | 26 | 18 |
| Example 2-A | 26 | 12 |
| Example 2-C | 26 | 9 |

The results as shown in Table 5 above were that Examples 2-A and 2-C had smaller rat populations having oily leakage as compared to Comparative Examples 1 and 2, because the porous adsorbent being empty after releasing the drug, had adsorbed free oil.

### TEST EXAMPLE 6

Oily Leakage Test using Human Model

Using 6 adult males with average weight of 70 kg as the subjects, one capsule (corresponds to 120 mg of orlistat) of Xenical was administered along with a single meal, and 7 days later, one pill (corresponds to 120 mg of orlistat) of the formulation prepared in Example 2-A was administered along with an identical meal.

The above-described meal consisted of a McDonald's Double Quarter Pounder with Cheese meal and a Chocolate Sundae ice cream, and it contained a total of 35 g of fat. Individual discomfort related to the anal discharge of oil or fat following drug administration was evaluated according to Table 6 and is shown in Table 7.

TABLE 6
Evaluation criteria

| Evaluation | |
|---|---|
| criteria | Discomfort related to oily anal leakage |
| 0 | Oily anal leakage is almost not sensed |
| 1 | Oily anal leakage is minor to the point that it does not |
| | negatively affect daily life |
| 2 | Oily anal leakage is severe to the point that it negatively |
| | affects daily life |

TABLE 7
Evaluation results

| Test subjects | 1 | 2 | 3 | 4 | 5 | 6 | Average |
|---|---|---|---|---|---|---|---|
| Comparative | 2 | 2 | 2 | 1 | 1 | 1 | 1.50 |
| Example 2 | | | | | | | |
| Example 2-A | 1 | 2 | 1 | 0 | 0 | 0 | 0.67 |

As a result shown in Table 7 above, Example 2-A was found to have had a lower incidence of oily anal leakage as compared to Comparative Example 2.

1. A method of preparing a pharmaceutical formulation for preventing and treating obesity which improves a dissolution rate and reduces side effect oily leakage, the method comprising:
forming a thin film of a lipase inhibitor on a porous adsorbent.

2. The method of claim 1, wherein the lipase inhibitor is selected from the group consisting of lipstatin, panclicins, hesperidin, ebelactones, esterastin, valilactone, orlistat, cetilistat, and derivatives and pharmaceutically acceptable salts thereof.

3. The method of claim 2, wherein the lipase inhibitor is orlistat or cetilistat.

4. The method of claim 1, wherein the thin film of the lipase inhibitor on the porous adsorbent is formed through melting or dissolution of the lipase inhibitor, followed by addition of the porous adsorbent.

5. The method of claim 1, wherein the thin film of the lipase inhibitor on the porous adsorbent is formed through mixing of the lipase inhibitor with the porous adsorbent, followed by melting or dissolution of the lipase inhibitor.

6. The method of claim 4 or 5, wherein the melting of the lipase inhibitor is performed through heating and pressurization in the presence of a supercritical fluid.

7. The method of claim 6, wherein the melting of the lipase inhibitor is performed at a temperature of 40 to 50° C. and a pressure of 90 to 110 bars.

8. The method of claim 4 or 5, wherein the melting of the lipase inhibitor is performed through heating to a temperature equal to or greater than a melting point of the lipase inhibitor.

9. The method of claim 4 or 5, wherein the dissolution of the lipase inhibitor is performed through dissolution of the lipase inhibitor in a volatile organic solvent.

10. The method of claim 9, wherein the volatile organic solvent is methanol, ethanol, acetone, acetonitrile, dichloromethanol, propanol, or a mixture thereof.

11. The method of claim 9, further comprising:
removing the volatile organic solvent after forming the thin film of the lipase inhibitor on the porous adsorbent.

12. The method of claim 4 or 5, wherein a non-volatile solvent, a solubilizer and a surfactant are not used in the melting or dissolution of the lipase inhibitor.

13. The method of claim 1, wherein the porous adsorbent is included at 1 to 50 parts by weight with respect to 1 part by weight of the lipase inhibitor.

14. The method of claim 1, wherein the porous adsorbent is magnesium aluminometasilicate, a zeolite, MCM-41, SBA-15, light anhydrous silicic acid, magnesium aluminosilicate, carbopol, a cellulose powder, crospovidone, sodium starch glycolate, croscarmellose sodium, carboxymethyl cellulose or a mixture thereof.

15. A pharmaceutical formulation for preventing and treating obesity which improves a dissolution rate and reduces side effect of oily leakage, the formulation comprising a lipase inhibitor and a porous adsorbent and having a thin film of the lipase inhibitor formed on the porous adsorbent.

16. The formulation of claim 15, wherein the lipase inhibitor is selected from the group consisting of lipstatin, panclicins, hesperidin, ebelactones, esterastin, valilactone, orlistat, cetilistat, and derivatives and pharmaceutically acceptable salts thereof.

17. The formulation of claim 16, wherein the lipase inhibitor is orlistat or cetilistat.

18. The formulation of claim 15, comprising:
the lipase inhibitor at 30 to 180 mg.

19. The formulation of claim 15, comprising:
the porous adsorbent at 1 to 50 parts by weight with respect to 1 part by weight of the lipase inhibitor.

20. The formulation of claim 15, wherein the porous adsorbent is magnesium aluminometasilicate, a zeolite, MCM-41, SBA-15, light anhydrous silicic acid, magnesium aluminosilicate, carbopol, a cellulose powder, crospovidone, sodium starch glycolate, croscarmellose sodium, carboxymethyl cellulose or a mixture thereof.
